(19) United States
(12) Patent Application Publication
(10) Pub. No.: US 2002/0119954 A1
(43) Pub. Date: Aug. 29, 2002

(54) Composition containing stabilized ascorbic acid, and uses thereof

(75) Inventors: Isabelle Afriat, Clichy Cedex (FR); Florence Chanvin, Clichy Cedex (FR); Sylviane Langlois, Clichy Cedex (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, s.a., Clichy Cedex (FR)

(21) Appl. No.: 09/988,132

(22) Filed: Nov. 19, 2001

Related U.S. Application Data
(63) Continuation of application No. 09/225,832, filed on Jan. 6, 1999, now abandoned.

(51) Int. Cl. A61K 31/375; A61K 31/695

(52) U.S. Cl. 514/63; 514/474

(57) ABSTRACT

The invention relates to a composition containing ascorbic acid and an oily phase, characterized in that the oily phase contains a partially or totally crosslinked, solid elastomeric organopolysiloxane. The presence of the solid elastomeric organopolysiloxane makes it possible to obtain good stability of the ascorbic acid and thus good efficacy of the composition containing it. The composition can be anhydrous or be in the form of a water-in-oil or oil-in-water emulsion or a triple emulsion. It can be used in particular in the cosmetic and/or dermatological fields. The presence of the solid elastomeric organopolysiloxane also makes it possible to stabilize an emulsion containing ascorbic acid.

### BACKGROUND OF THE INVENTION

1. Field of the Invention

The invention relates to a composition containing stabilized ascorbic acid and an oily phase, as well as to a use of this composition in the cosmetic and/or dermatological fields, and to a process for treating human skin using such a composition.

The invention also relates to the use of a partially or totally crosslinked, solid elastomeric organopolysiloxane to stabilize ascorbic acid.

2. Discussion of the Background

It has long been sought to formulate ascorbic acid (or vitamin C) in the cosmetic and dermatological fields, in different pharmaceutical forms, on account of its many beneficial properties. In particular, ascorbic acid stimulates the synthesis of connective tissue and in particular of collagen, strengthens skin tissue defences against external attacking factors such as ultraviolet radiation and pollution, compensates for vitamin E deficiency in the skin, depigments the skin and has an anti-free-radical function. On account of its properties, ascorbic acid is effective at cleaning the skin and also at combating the signs of ageing of the skin, for example it improves the radiance of the complexion and smooths out wrinkles and/or fine lines on the sin.

Unfortunately, on account of its chemical structure (α-keto lactone), ascorbic acid is very sensitive to certain environmental parameters such as light, oxygen and water. Rapid degradation of formulated ascorbic acid thus follows when it is in contact with one of these parameters in particular, which is an obstacle to the desired efficacy.

In order to reduce and/or delay the degradation of ascorbic acid, several solutions have already been envisaged in the prior art. Thus, document U.S. Pat. No. 5,140,043 recommends stabilizing it by introducing it into aqueous-alcoholic solutions formed of at least 50% water and having a pH of less than 3.5. On account of the high acidity of these solutions, their use in the cosmetic and/or pharmaceutical field cannot be readily envisaged, since repeated application of these solutions can disrupt the skin's equilibrium and in particular irritate or even burn the skin. In addition, at acidic pH, although the color change is stabilized, the generation of carbon dioxide (CO₂) is not.

Other ways of stabilizing ascorbic acid have been envisaged, in particular by coating (technique described in document FR-A-1,600,826) or by granulating ascorbic acid (technique illustrated in document JP-A-53-127,819, for the agrifoods sector). However, these techniques are expensive and are not always suitable for topical application.

Moreover, it is known, from document FR-A-1,489,249, to use metal salts of phosphorylated ascorbic acid, in particular magnesium ascorbyl phosphate, in cosmetic compositions. Such derivatives are often less efficient than ascorbic acid in free form.

Moreover, document EP-A-755,674 describes compositions containing a large amount of polyol associated with a polymer or an oil, in which the ascorbic acid is stable. However, the use of a large amount of polyol can have certain drawbacks, such as a lack of comfort when applied to the skin.

There is thus a need for a composition which can be used in particular in the cosmetic and/or dermatological fields, containing stabilized ascorbic acid in free form, i.e. without any additional group, in particular stabilizer, this composition being comfortable when applied and not causing any skin irritation after application.

The Applicant has now found a composition which overcomes the drawbacks of the prior art.

### SUMMARY OF THE INVENTION

The subject of the present invention is a composition containing ascorbic acid and an oily phase, characterized in that the oily phase contains at least one partially or totally crosslinked, solid elastomeric organopolysiloxane.

The term “elastomer” is understood to refer to a flexible, deformable material having viscoelastic properties and in particular the consistency of a sponge or a flexible sphere.

By virtue of the presence of elastomeric organopolysiloxanes in the composition of the invention, the ascorbic acid is more stable and thus more effective than in a composition not containing any.

Thus, another subject of the present invention is the use of at least one partially or totally crosslinked, solid elastomeric organopolysiloxane to stabilize ascorbic acid.

The ascorbic acid can be of any nature. Thus, it can be of natural origin in powder form or in the form of orange juice, preferably concentrated. It can also be of synthetic origin, preferably in powder form.

The ascorbic acid concentrations in the composition of the invention are those used conventionally in the fields considered, and, for example, from 0.1 to 20%, and preferably from 0.5 to 10%, of the total weight of the composition.

The elastomeric organopolysiloxanes used in the composition according to the invention are partially or totally crosslinked. When included in an oily phase, they become converted, depending on the level of oily phase used, from a product of spongy appearance when they are used in the presence of low amounts of oily phase, into a homogeneous gel in the presence of larger amounts of oily phase. The gelation of the oily phase by these elastomers may be total or partial.

The elastomers of the invention can be conveyed in the form of a gel consisting of an elastomeric organopolysiloxane, including at least one hydrocarbon oil and/or a silicone oil and/or a fluoro oil. Thus, the oily phase associated with the elastomeric organopolysiloxane can consist of this or these oils.

The elastomeric organopolysiloxanes used according to the invention can be chosen from the crosslinked polymers described in patent application EP-A-0,295,886; incorporated herein by reference. According to that application, they are obtained by an addition and crosslinking reaction, in the presence of a platinum-type catalyst, of at least:

(a) one organopolysiloxane having at least two lower alkenyl groups per molecule, these alkenyl groups containing two to six carbon atoms; and

(b) one organopolysiloxane having at least two hydrogen atoms linked to a silicon atom per molecule.

The elastomeric organopolysiloxanes used in the composition of the invention can also be chosen from those described in patent U.S. Pat. No. 5,266,231; incorporated herein by reference. According to that patent, they are chosen in particular from:

i) organopolysiloxanes comprising units R₂SiO and RSiO_{1.5} and optionally units R₃SiO_{0.5} and/or SiO₂, in which the radicals R, independently of each other, denote a hydrogen, an alkyl radical such as methyl, ethyl or propyl, an aryl radical such as phenyl or tolyl, an unsaturated aliphatic group such as vinyl, the weight ratio of the units R₂SiO to the units RsiO_{1.5} ranging from 1/1 to 30/1;

ii) organopolysiloxanes which are insoluble and swellable in silicone oil, obtained by addition of an organohydrogenopolysiloxane (1) and of an organopolysiloxane (2) having unsaturated aliphatic groups, such that the amount of hydrogen or of unsaturated aliphatic groups in (1) and (2) respectively is between 1 and 20 mol % when the organopolysiloxane is non-cyclic and between 1 and 50 mol % when the organopolysiloxane is cyclic.

The organopolysiloxanes in the composition of the invention are, for example, those sold under the names KSG 6 from Shin-Etsu, Trefil E-505C or Trefil E-506C from Dow-Corning, Gransil (SR-CYC, SR DMF10, SR DC556) from Grant Industries, or those sold in the form of preconstituted gels: KSG 15, KSG 17, KSG 16, KSG 18, KSG 26A and KSG 26B from Shin-Etsu, Gransil SR 5CYC gel, Gransil SR DMF 10 gel and Gransil SR DC556 gel from Grant Industries, and 1229-02-167 and 1229-02-168 from General Electric. A mixture of these commercial products can also be used.

The elastomeric organopolysiloxanes used according to the invention have the advantage not only of stabilizing ascorbic acid but also of not drying out the skin and of providing good cosmetic properties. They lead to compositions which are comfortable when applied and which feel soft and non-sticky. This softness is due in particular to the texture of the organopolysiloxanes.

Preferably, the elastomeric organopolysiloxane(s) used according to the invention is (are) present in an active material concentration ranging from 0.1 to 20%, preferably from 0.5 to 15% and better still from 1.5 to 15%, of the total weight of the composition. However, these proportions of organopolysiloxane as well as those of the oily phase, can vary depending on the pharmaceutical form which it is desired to obtain.

Preferably, the oil constituting the vehicle of the elastomeric organopolysiloxane comprises one or more hydrocarbon oils and/or silicone oils and/or fluoro oils. These oils can be volatile or non-volatile and are chosen as a function of their solubility parameters and their chemical structure. The oil used or the mixture of oils used preferably has average Hansen solubility parameters dD, dP and dH at 25° C. which satisfy the following three conditions:

(1) dD≦20 (J/cm³)^{½}

(2) dP≦10 (J/cm³)^{½}

(3) dH≦15 (J/cm³)^{½.}

The definition of the solvents in the three-dimensional solubility space according to Hansen is described in the article by C. M. Hansen: “The three-dimensional solubility parameters” J. Paint Technol. 39, 105 (1967). This space is defined by the parameters dD, dP, dH; they are expressed in (J/cm³)^{½:}

dD characterizes the London dispersion forces derived from the formation of dipoles induced during molecular impacts;

dP characterizes the Debye interaction forces between permanent dipoles and the Keesom interaction forces between induced dipoles and permanent dipoles;

dH characterizes the specific forces of interaction (of the hydrogen bonding, acid/base, donor/acceptor, etc. type).

The hydrocarbon oils can be chosen from oils of animal origin, oils of plant origin, synthetic oils such as hydrogenated isoparaffin, synthetic esters and ethers, and mixtures thereof.

The silicone oils can be chosen from linear polysiloxanes which are liquid or pasty at room temperature, such as polydimethylsiloxanes, alkylpolysiloxanes, alkylphenylpolysiloxanes, alkylpolydimethylsiloxanes, and cyclic polysiloxanes such as octamethylcyclopentasiloxane and decamethylcyclopentasiloxane, or mixtures thereof.

The oily phase of the composition of the invention can consist of any oil and in particular the oils mentioned above.

The composition can be used as it is in anhydrous form, but an aqueous phase can also be incorporated therein in order to obtain a water-in-oil (W/O) emulsion, an oil-in-water (O/W) emulsion or alternatively a triple emulsion such as a water-in-oil-in-water (W/O/W) or oil-in-water-in-oil (O/W/O) emulsion. The term “emulsion” is understood here to refer both to emulsifier free dispersions and to dispersions containing emulsifiers, or alternatively dispersions stabilized with lipid spherules of ionic or nonionic type.

In the anhydrous composition, the oily phase, including the amount of the elastomeric organopolysiloxane(s), is present in a concentration ranging from 80 to 99.9% and preferably from 90 to 99.5% of the total weight of the composition.

In the compositions of the invention in emulsion form, the aqueous phase of the composition is present in a concentration ranging from 1 to 60%, and preferably 20 to 50%, of the total weight of the composition, and the oily phase, including the amount of the elastomeric organopolysiloxane(s), is present in a concentration ranging from 20 to 98.9%, and preferably from 30 to 60%, of the total weight of the composition.

The presence of the solid elastomeric organopolysiloxane makes it possible not only to stabilize the ascorbic acid, i.e. to retain its activity, but also to stabilize the emulsion containing it, i.e. to avoid a separation of the phases of the emulsion, since emulsions containing ascorbic acid have a tendency to become destabilized on account of the presence of this acidic active agent. The invention allows this drawback to be overcome.

Thus, another subject of the present invention is the use of at least one partially or totally crosslinked, solid elastomeric organopolysiloxane to stabilize an emulsion containing ascorbic acid.

When it is in emulsion form, the composition of the invention preferably contains at least one emulsifier. The nature of the emulsifier(s) used depends on the type of emulsion which it is desired to obtain. For example, a silicone-containing emulsifier such as a dimethyiconecopolyol and/or an alkyldimethiconecopolyol can be used.

For the W/O emulsions, as silicone-containing emulsifier, mention may be made of the mixture of dimethiconecopolyol and cyclomethicone sold under the name “Q2-3225C” by the company Dow Corning, the product sold under the name “SF 1228” by the company General Electric, lauryldimethiconecopolyol sold under the name “Q2-5200” by the company Dow Corning and cetyldimethiconecopolyol sold under the name “Abil EM 90” by the company Goldschmidt.

As an emulsifier for O/W emulsions, mention may be made, for example, of oxyethylenated polydimethylmethylsiloxane (dimethiconecopolyol) sold under the name “DC2-5695” by the company Dow Corning.

The emulsifier(s) can be present in the composition according to the invention in a concentration which can vary within a wide range. Thus, this concentration can range, for example, from 0.1 to 20%, and preferably from I to 5%, of the total weight of the composition.

Advantageously, and in order in particular to avoid the presence in the aqueous phase of heavy metals which might catalyze the degradation of the ascorbic acid, the aqueous phase contains exchanged or deionized water.

In order to further increase the stability of ascorbic acid over time, the composition of the invention can also advantageously comprise at least one compound chosen from metal-sequestering agents, metabisulphites and polyols.

The metal-sequestering agent can in particular be a phosphonic acid derivative. As phosphonic acid derivatives which can be used in the composition of the invention, mention may be made in particular of ethylenediaminetetra(methylenephosphonic acid), hexamethylenediaminetetra(methylenephosphonic acid), diethylenetriaminepenta(methylenephosphonic acid) and salts thereof, in particular the sodium salts thereof, such as pentasodium ethylenediaminetetra(methylenephosphonate).

Advantageously, ethylenediaminetetra(methylenephosphonic acid) sold in particular by the company Monsanto under the name Dequest 2041 and the pentasodium salt of this acid, sold under the name Dequest 2046 by the company Monsanto, are used.

When it is present, the sequestering agent is in a concentration generally ranging from 0.05 to 0.5% by weight relative to the total weight of the emulsion.

The metabisulphite can be an alkali-metal, alkaline-earth metal or ammonium salt of anhydrosulphurous acid. Sodium or potassium metabisulphite is preferably used. When it is present, the metabisulphite is in a concentration generally ranging from 0.005 to 5%, and preferably from 0.05 to 1%, of the total weight of the composition.

The polyols which may be present in the composition of the invention can be chosen, from example, from glycerol, glycols such as propylene glycol and polyethylene glycol, and silicones containing hydroxyl groups. The polyols are present in a concentration preferably ranging from 0.5 to 30%, and more preferably from 5 to 25%, of the total weight of the composition.

The composition of the invention can be used in particular in topical application in the cosmetic and dermatological fields. For topical and in particular cosmetic and/or dermatological application, the composition according to the invention must contain a physiologically acceptable medium, i.e. one which is compatible with human skin, mucous membranes and/or hair. This composition can be more or less fluid and have the appearance of a white or colored cream, an ointment, a milk, a lotion, a serum, a paste or a mousse.

The composition of the invention can be applied topically to the human face, including the area around the eyes, the body and the scalp.

In a known manner, the composition of the invention can also contain additives that are common in the cosmetic and dermatological fields, such as hydrophilic or lipophilic active agents other than ascorbic acid, preserving agents, antioxidants, solvents, fragrances, fillers, odor absorbers and dyestuffs, provided that the additive does not destabilize the ascorbic acid in the emulsion. The amounts of these various additives are those used conventionally in the fields considered, and, for example, from 0.01 to 20% of the total weight of the composition. Depending on their nature, these additives can be introduced into the oily phase or into the aqueous phase.

As hydrophilic gelling agents, mention may be made in particular of carboxyvinyl polymers (carbomer), acrylic copolymers such as acrylate/alkylacrylate copolymers, polyacrylamides, polysaccharides, natural gums and clays, and as lipophilic gelling agents, mention may be made of modified clays such as bentones, metal salts of fatty acids, hydrophobic silica and polyethylenes. The product sold under the name Sepigel 305 by the company SEPPIC (CTFA name polyacrylamide/C13-14 isoparaffin/laureth-7) and the product sold under the name Hostacerin AMPS by the company Hoechst (CTFA name: ammonium polyacryldimethyltauramide) can be used in particular as gelling agents.

Moisturizers such as the polyols mentioned above, proteins or protein hydrolysates, sodium pyrrolidone carboxylate, NMFs (normal moisturizing factors), hyaluronic acid, amino acids, allantoin, sugars (glucose, mannose) and derivatives thereof, vitamin E and derivatives thereof, essential fatty acids, ceramides, essential oils, keratolytic and/or desquamating agents salicylic acid and derivatives thereof, a-hydroxy acids), antiinflammatory agents, UV screening agents and calmants, and mixtures thereof, can be used in particular as active agents. In case of incompatibility, these active agents can be incorporated into spherules, in particular ionic or nonionic vesicles and/or nanoparticles (nanocapsules and/or nanospheres) in order to isolate them from each other in the composition.

As already mentioned above, the degradation of ascorbic acid, however minimal the amount, leads to yellowing of the composition containing it. For this reason, it is preferable, in order to avoid this yellowing, for the composition according to the invention to be packaged such that it is not in contact with oxygen and is protected from the light. Thus, the composition of the invention is preferably prepared under inert atmosphere (nitrogen or a rare gas such as argon), which is free of all oxygen or contains less than 5% v/v of oxygen, and under inactinic light, such as that of a sodium vapor lamp.

In addition, advantageously, the composition of the invention can be packaged in the presence of an oxygen absorber. Even more preferably, the composition of the invention is packaged in a container on which is mounted a distribution device without an air return, such as, for example, the one described in document FR-A-2,666,308; incorporated herein by reference.

The composition of the invention has good efficacy in all the applications of vitamin C, especially for cleaning skin and/or treating it, in particular for toning or regenerating it, for treating wrinkles and/or fine lines on the skin, for lightening the complexion, for removing skin pigmentation marks, for combating the harmful effects of UV radiation and/or for strengthening skin tissue against environmental attack.

Thus, the subject of the present invention is also the cosmetic use of the composition according to the invention for cleaning skin and/or treating it, in particular for toning or regenerating it, for treating wrinkles and/or fine lines on the skin, for lightening the complexion, for removing skin pigmentation marks, for combating the harmful effects of UV radiation and/or for strengthening skin tissue against environmental attack.

The subject of the invention is also the use of the composition according to the invention for the manufacture of a cream which is intended for a dermatological skin treatment.

Lastly, the subject of the invention is a cosmetic skin cleaning and/or treatment process, which consists in applying a composition in accordance with the invention to the skin, including the area around the eyes.

Other advantages and characteristics of the invention will become more apparent on reading the examples given by way of non-limiting illustration.

### EXAMPLE 1

### Face Cream (W/O Emulsion)

Oily phase:

| | |
|---|---|
| Mixture of dimethiconecopolyol and of | 20% |
| cyclomethicone (Q2-3225C from Dow corning) | |
| Phenyltrimethicone (Dow Coming 556 fluid) | 4% |
| Apricot oil | 3% |
| Crosslinked organopolysiloxane containing | 8% |
| 24% active material in non-volatile PDMS (KSG 16) | |

Aqueous phase:

| | |
|---|---|
| Glycerol | 23% |
| Propylene glycol | 6% |
| Sodium hydroxide | 1.8% |
| Citric acid | 1.2% |
| Ascorbic acid | 5% |
| Deionized water | qs |
| | 100% |

The emulsion is prepared by preparing the phases and introducing the aqueous phase into the oily phase with stirring while cold.

The composition obtained is in the form of a cream which is suitable for facial care and is soft when applied. This cream gives an immediately radiant complexion and allows imperfections to be smoothed out.

Good stability of the ascorbic acid in the emulsion and good stability of the emulsion itself were observed.

### EXAMPLE 2

### Care Cream (O/W Emulsion)

Oily phase:

| | |
|---|---|
| Dimethiconecopolyol (DC2-5695) | 2% |
| Plant oil | 5% |
| Crosslinked organopolysiloxane containing | 4% |
| 24% active material in non-volatile PDMS (KSG 16) | |
| Polydimethylsiloxane (10 cSt) | 11% |
| Fragrance | 0.3% |
| Hydrogenated polyisobutene | 5% |

Aqueous phase:

| | |
|---|---|
| Polyethylene glycol | 3% |
| Glycerol | 8% |
| Sepigel 305 | 4% |
| Hostacerin AMPS | 2% |
| Sodium metabisulphite | 0.05% |
| Dequest 2041 | 0.1% |
| Glucose | 0.5% |
| Sodium hydroxide | 2% |
| Citric acid | 1.2% |
| Ascorbic acid | 5% |
| Preserving agent | 0.1% |

Deionized water qs 100%

The emulsion is prepared by preparing the phases and introducing the oily phase into the aqueous phase with stirring while cold.

A cream capable of making the complexion radiant and of smoothing out the fine lines on the skin is obtained.

### EXAMPLE 3

### Face Cream (W/O Emulsion)

Example 3 is identical to Example 1, with, however, a KSG 16 content of 5% instead of 8%.

Other preferred embodiments include compositions comprising, consisting essentially of, and consisting of all combinations of ascorbic acid with any one or more of the components described above.

Where numerical ranges are described herein, they are inclusive of endpoints and specifically refer to and include all numbers and subranges therewithin, thereby identifying each and every number and subrange within the broadest stated range.

Particularly preferred embodiments include the following:

A. Composition containing ascorbic acid and an oily phase, characterized in that the oily phase contains at least one partially or totally crosslinked, solid elastomeric organopolysiloxane.

B. Composition according to A, characterized in that the elastomeric organopolysiloxane is obtained by an addition and crosslinking reaction, in the presence of a catalyst, of at least:

(a) one organopolysiloxane having at least two lower alkenyl groups per molecule;

(b) one organopolysiloxane having at least two hydrogen atoms linked to asilicon atom per molecule.

C. Composition according to A or B, characterized in that the organopolysiloxane is chosen from:

i) organopolysiloxanes comprising units R₂SiO and RSiO_{1.5} and optionally units R₃SiO_{0.5} and/or SiO₂, in which the radicals R, independently of each other, denote a hydrogen, an alkyl radical, an aryl radical, an unsaturated aliphatic group, the weight ratio of the units R₂SiO to the units RSiO_{1.5} ranging from 1/1 to 30/1;

ii) organopolysiloxanes which are insoluble in silicone oil, obtained by addition of an organohydrogenopolysiloxane (1) and of an organopolysiloxane (2) having unsaturated aliphatic groups, such that the amount of hydrogen or of unsaturated aliphatic groups in (1) and (2) respectively is between 1 and 20 mol % when the organopolysiloxane is non-cyclic and between 1 and 50 mol % when the organopolysiloxane is cyclic.

D. Composition according to any one of the preceding embodiments, characterized in that the solid elastomeric organopolysiloxane is present in an active material concentration ranging from 0.1 to 20%, and preferably from 0.5 to 15%, of the total weight of the composition.

E. Composition according to any one of the preceding embodiments, characterized in that the oily phase also comprises at least one oil chosen from hydrocarbon oils, silicone oils, fluoro oils and mixtures thereof.

F. Composition according to the preceding embodiment, characterized in that the hydrocarbon oil is chosen from oils of animal origin, oils of plant origin, synthetic oils and mixtures thereof.

G. Composition according to E, characterized in that the silicone oil is chosen from linear polysiloxanes which are liquid or pasty at room temperature, cyclic polysiloxanes and mixtures thereof.

H. Composition according to any one of the preceding embodiments, characterized in that the oily phase is present in a concentration ranging from 80 to 99.9% of the total weight of the composition.

I. Composition according to any one of the preceding embodiments, characterized in that the ascorbic acid is present in a concentration ranging from 0.1 to 20% of the total weight of the composition.

J. Composition according to any one of the preceding embodiments, characterized in that it also comprises an aqueous phase.

K. Composition according to any one of the preceding embodiments, characterized in that it is a water-in-oil emulsion, an oil-in-water emulsion or a triple emulsion.

L. Composition according to either of J and K, characterized in that the aqueous phase represents from 1 to 60%, and preferably 20 to 50%, of the total weight of the composition.

M. Composition according to any one of J-L, characterized in that the oily phase represents from 20 to 98.9%, and preferably from 30 to 60%, of the total weight of the composition.

N. Composition according to any one of J-M, characterized in that it also comprises an emulsifier.

O. Composition according to N, characterized in that the emulsifier is present in a proportion of from 0.1 to 20% of the total weight of the composition.

P. Composition according to N or 0, characterized in that the emulsifier is a silicone-containing emulsifier.

Q. Composition according to any one of the preceding embodiments, characterized in that it also comprises at least one compound chosen from metal-sequestering agents, metabisulphites and polyols.

R. Composition according to any one of the preceding embodiments, characterized in that it constitutes a topical-application composition.

S. Composition according to any one of the preceding embodiments, characterized in that it also comprises at least one additive chosen from hydrophilic or lipophilic active agents, preserving agents, antioxidants, solvents, fragrances, fillers, odor absorbers and dyestuffs.

T. Use of at least one partially or totally crosslinked, solid elastomeric organopolysiloxane to stabilize ascorbic acid.

U. Use according to the preceding embodiment, characterized in that the elastomeric organopolysiloxane is in an oily phase.

V. Use of at least one partially or totally crosslinked, solid elastomeric organopolysiloxane to stabilize an emulsion containing ascorbic acid.

W. Cosmetic use of a composition according to any one of A-S, for cleaning skin and/or treating it, in particular for toning or regenerating it, for treating wrinkles and/or fine lines on the skin, for lightening the complexion, for removing skin pigmentation marks, for combating the harmful effects of UV radiation and/or for strengthening skin tissue against environmental attack.

X. Use of a composition according to any one of A-S, for the manufacture of a cream which is intended for a dermatological skin treatment.

Y. Cosmetic skin cleaning and/or treatment process, characterized in that it consists in applying a composition according to any one of A-S to the skin, including the area around the eyes.

We claim:

1. A method of stabilizing ascorbic acid, comprising combining ascorbic acid and at least one partially or totally crosslinked, solid elastomeric organopolysiloxane.

2. A composition comprising ascorbic acid, at least one partially or totally crosslinked, solid elastomeric organopolysiloxane, and an oil.

3. The composition of claim 2, wherein said oil is a cyclic polysiloxane.

4. The composition of claim 2, wherein said oil is decamethylcyclopentasiloxane.
